(19) United States
(12) Patent Application Publication
(10) Pub. No.: US 2010/0204292 A1
(43) Pub. Date: Aug. 12, 2010

(54) Pharmaceutical compositions comprising intra-and extra-granular fractions

(76) Inventors: Jack Aurora, Grand Rapids, MI (US); Naresh Talwar, Kirkland (CA); Jyoti Missra, Dollard-Des-Ormeaux (CA); Kiran Shankar Lagu, Vaudreuil-Dorion (CA)

Correspondence Address: STIKEMAN ELLIOTT LLP, 1600-50 O''CONNOR STREET, OTTAWA, ON, K1P 6L2, CA

(21) Appl. No.: 12/311,700

(22) PCT Filed: Aug. 17, 2007
(86) PCT No.: PCT/CA2007/001449

§ 371 Date: Apr. 14, 2010

(30) Foreign Application Priority Data
Oct. 12, 2006 (CA) 2,563,690

(51) Int. Cl. A61K 31/4184

(52) U.S. Cl. 514/381

(57) ABSTRACT

A pharmaceutical composition comprising i) an intra-granular fraction containing a pharmaceutically acceptable active component and a first excipient and ii) an extra-granular fraction containing a second excipient. The pharmaceutical composition is characterized by being substantially surfactant-free and by exhibiting a rapid-dissolution profile.

This is a National Phase application based on International Application Serial No. PCT/CA2007/001449 filed Aug. 17, 2007, which claims priority of Canadian Patent Application No. 2,563,690, filed Oct. 12, 2006.

The present invention relates to pharmaceutical formulations or compositions for oral administration of a pharmaceutically acceptable active component or element; the pharmaceutically acceptable active component or element may for example comprise (or consists of) irbesartan or a pharmaceutically acceptable salt thereof. The present invention in particular relates to pharmaceutical compositions comprising an intra-granular fraction and an extra-granular fraction. The present invention also relates to pharmaceutical formulations or compositions in the (unit) dosage form (e.g. tablets).

The present invention will be described hereinafter, by way of example, in relation to irbesartan.

Thus it is known, for example, to provide irbesartan containing pharmaceutical compositions which in addition to the pharmaceutically acceptable active element comprises a diluent and a surfactant; see U.S. Pat. No. 6,342,247 the entire contents of which are incorporated herein by reference. It is also known to associate Irbesartan with other pharmaceutically active substances, such as a diuretic (see U.S. Pat. No. 6,342,247).

Irbesartan, (i.e. 2-n-butyl-4-spirocyclopentane-1-[(2′-(tetrazol-5-yl)biphenyl-4-yl)methyl]-2-imidazolin-5-one) is useful in the treatment of cardiovascular ailments such as hypertension and heart failure. Irbesartan is described in U.S. Pat. No. 5,270,317, (the entire contents of which are incorporated herein by reference) and has the following structure:

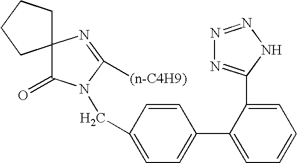

Irbesartan may also be used in a pharmaceutically acceptable salt form, e.g. alkali and alkaline earth metal salts (e.g. Na, K, etc), sesquihydrate hydrochlorite salt, anhydrous hydrochlorite salt and nitric acid salts (see; for example U.S. Pat. No. 6,342,247, CA 2,536,781, WO 2006/050923 and WO 2006/011859 for salts).

It is known that Irbesartan may be administered in doses from 75 to 300 mg. Certain physical properties of this drug present a challenge in developing formulations suitable for preparing a tablet.

Irbesartan is, for example, a fluffy, relatively sticky material, with relatively low bulk density.

There is a continuing need for pharmaceutical compositions or formulations (containing a pharmaceutically active component or element such as for example irbesartan), which not only have properties suitable for tablet formation, but which also facilitate relatively rapid dissolution and drug release.

Thus, the present invention in a general aspect relates to a pharmaceutical composition comprising a pharmaceutically acceptable active component and a pharmaceutically acceptable excipient component, said excipient component comprising a dissolution modifying excipient element, said composition being characterized in that the composition (i.e. the excipient component) is at least substantially surfactant free (i.e. free or substantially free of surfactant).

The present invention in particular aspect relates to a pharmaceutical composition comprising a pharmaceutically acceptable active component which comprises or consists of Irbesartan (or a pharmaceutically acceptable salt thereof) and a pharmaceutically acceptable excipient component, said excipient component comprising a dissolution modifying excipient element, said composition being characterized in that the composition (i.e. the excipient component) is at least substantially surfactant free (i.e. free or substantially free of surfactant).

In accordance with the present invention there is thus provided a pharmaceutical composition comprising

an intra-granular fraction (or portion) intermingled

with an extra-granular fraction (portion),

said intra-granular fraction consisting of granules comprising a (infra-granular) pharmaceutically acceptable active component and a first pharmaceutically acceptable excipient component, said first pharmaceutically acceptable excipient component comprising (or consisting of) a (first or intra-granular) binder element, a first disintegrant element and a first anti-adherent element, said intra-granular fraction being at least substantially free of a diluent element and being at least substantially free of a surfactant, element, said extra-granular fraction comprising (or consisting of) a second pharmaceutically acceptable excipient component, said second pharmaceutically acceptable excipient component comprising (or consisting of) a diluent element, (optionally, (i.e. as desired or necessary), a second binder element), a second disintegrant element, a second anti-adherent element and a lubricant element, said extra-granular fraction being at least substantially free of a surfactant element.

A pharmaceutical composition in accordance with the present invention may not only take the form of a mixture or blend of an intra-granular fraction and an extra-granular fraction (as described herein) but may, for example, also, once having been subjected to a suitable compression technique, take on a (unit) dosage form (e.g. tablet).

Thus, the present invention further relates to a tablet prepared from the compression of a tablet formulation (i.e. pharmaceutical composition) comprising a mixture or blend of

an intra-granular fraction (or portion) and

an extra-granular fraction (or portion),

said intra-granular fraction consisting of granules comprising (or consisting of) a (intra granular) pharmaceutically acceptable active component and a first pharmaceutically acceptable excipient component, said first pharmaceutically acceptable excipient component comprising (or consisting of) a (first or intra-granular) binder element, a first disintegrant element and a first anti-adherent element, said intra-granular fraction being at least substantially free of a diluent element and being at least substantially free of a surfactant element, said extra-granular fraction comprising (or consisting of) a second pharmaceutically acceptable excipient component, said second pharmaceutically acceptable excipient component comprising (or consisting of) a diluent element, (optionally, (i.e. as desired or necessary), a second binder element), a second disintegrant element, a second anti-adherent element and a lubricant element, said extra-granular fraction being at least substantially free of a surfactant element.

The present invention in particular provides a pharmaceutical composition (or dosage form such as, for example, a tablet) wherein the medicament (i.e. the (first and second) pharmaceutically acceptable active component) may in particular comprise a member selected from the group consisting of irbesartan and pharmaceutically acceptable salts thereof.

It is to be understood herein that the words diluent, binder, surfactant, disintegrant, anti-adherent, lubricant, etc. are used herein as adjectives to categorize or describe elements (e.g. substance(s) or compound(s)) of a pharmaceutical composition in relation to an element's function as part of such pharmaceutical composition.

It is to be understood herein that a “surfactant element” for the present invention may be any of one or more (known) substances or compounds which (in appropriate amounts) are capable of providing a surfactant effect i.e. capable of modifying (e.g. improving or enhancing) the wetting and/or solubility characteristics of a drug containing dosage form (e.g. tablet). For the purposes of the present invention, a “surfactant element” is to be avoided, as described herein.

It is to be understood herein that the expressions “at least substantially surfactant free”, “at least substantially free of surfactant element” or the like are meant to characterize a pharmaceutical composition (or formulation or tablet) of the present invention as well as fractions or components thereof as not comprising any surfactant substance(s) or as not comprising any significant amount(s) of surfactant substance(s); in other words, for example, there is no compound(s) or substance(s) added (i.e. admixed) to the other elements of the composition which is able to provide a surfactant effect or if such a compound(s) or substance(s) is present it is not present in an amount sufficient to give rise to an undesirable surfactant effect, for example an amount less than 0.01% (e.g. less than 0.0001%) by weight of the composition (or tablet) mass.

It is to be understood herein that the expression “at least substantially free of diluent element” or the like is meant to characterize the intra-granular fraction or components of a pharmaceutical composition (or formulations or tablet) as not comprising a diluent or filler type substance(s) or compound(s) which are exploited to alter or modify the mass of an obtained composition (e.g. tablet) or if such a compound(s) or substance(s) is present it is not present in an amount sufficient to give rise to the exploitation thereof for such mass altering effect. On the other hand, it is nevertheless to be understood herein, that the above expressions with respect to the absence of a diluent element, do not necessarily exclude a substance(s) or compound(s) having a (known) diluent function but which may also be exploited so as to effectively take advantage of a different excipient function (e.g. as a binder, disintegrant, etc.) i.e. in other words such a dual purpose substance(s) or compound(s) may yet be present so as to effectively take advantage of such different excipient function (e.g. as a binder element, disintegrant element, etc.). However, such a dual purpose substance(s) or compound(s) may only be present provided that the (dissolution) characteristics of the resultant tablet are not undesirably affected thereby.

The present invention allows for the preparation of pharmaceutical compositions (e.g. (unit) dosage forms such as tablets) which have a favorable dissolution characteristic as compared to tablets made exploiting different formulations which contain surfactant material(s). Thus a tablet in accordance with the present invention may dissolve in 10 minutes as compared with other tablet types which may dissolve the same amount in 30 minutes.

It is to be understood herein that any reference to % by weight (i.e. % w/w) is a reference to weight in relation to the total weight of the composition (or formulation or tablet) mass unless indicated otherwise.

A pharmaceutical composition (e.g. tablet) of the present invention may suitably comprise high amounts of medicament so that a high proportion of medicament is present. For example, the pharmaceutically acceptable active component(s) or element(s) (e.g. irbesartan or a pharmaceutically acceptable salt thereof or a mixture thereof) may be present in an amount of at least 30% or more (e.g. up to 71%) of the total weight of the composition (or tablet), with the remainder comprising pharmaceutically acceptable excipient substance(s) or material(s)) present for example in an amount of from 70% or less (e.g. from 29% to 70%) of the total weight of the composition (or tablet). If desired or necessary, lower amounts of medicament may, of course, be present in the pharmaceutical composition, i.e. with correspondingly higher amounts of excipient substance(s) or material(s). Herein the amount of excipient substance(s) or material(s) will in the usual course be a function of the amount of medicament desired to be present in the composition (e.g. tablet), i.e. the amount of medicament in the usual course will take precedence over the amount(s) of excipient substance(s) or material(s), with the amount of the various excipient substance(s) or material(s) being adjusted as necessary or desired in view of the desired amount of medicament.

The intra-granular fraction (i.e. granules) may for example comprise up to 75% of the total weight of the composition (or tablet), e.g. from 40% to 75% by weight of the composition (or tablet). The (first) binder element of the intra-granular fraction may for example comprise up to 20% of the total weight of the composition (or tablet), e.g. from 0.5% to 20% by weight of the composition (or tablet). The (first) disintegrant element of the intra-granular fraction may for example comprise up to 15% of the total weight of the composition (or tablet), e.g. from 0.1% to 15% by weight of the composition (or tablet). The (first) anti-adherent element of the intra-granular fraction may comprise up to 5% of the total weight of the composition (or tablet), e.g. from 0.01% to 5% by weight of the composition (or tablet).

The extra-granular fraction may for example comprise up to 60% of the total weight of the composition (or tablet), e.g. from 25% to 60% by weight of the composition (or tablet). The diluent element of the extra-granular fraction may for example comprise up to 59.6% of the total weight of the composition (or tablet), e.g. from 10% to 59.6% by weight of the composition (or tablet). The (second) disintegrant element of the extra-granular fraction may for example comprise up to 15% of the total weight of the composition (or tablet), e.g. from 0.1% to 15% by weight of the composition (or tablet). The (second) binder element, if present, of the extra-granular fraction, may be present per se or for the granulation of the diluent where required; the second binder element may thus, for example, be present in an amount of from 0 up to 10. % of the total weight of the composition (or tablet), e.g. from 0.5% to 10% by weight of the composition (or tablet). The (second) anti-adherent element of the extra-granular fraction may for example comprise up to 5% of the total weight of the composition (or tablet), e.g. from 0.1% to 5% by weight of the composition (or tablet). The lubricant element of the extra-granular fraction may for example comprise up to 7.5% of the total weight of the composition (or tablet), e.g. from 0.2% to 7.5% by weight of the composition (or tablet).

The intra-granular fraction may, alone, comprise a pharmaceutically acceptable active component, (i.e. an intra-granular or first pharmaceutically acceptable active component). Thus, the extra-granular fraction may comprise no pharmaceutically acceptable active component or element. Alternatively, the extra-granular fraction may comprise some pharmaceutically acceptable active component provided that the (dissolution) characteristics of the resultant tablet (i.e. resultant composition) are not undesirably affected thereby. Thus the extra-granular fraction may comprise a (second) pharmaceutically active component in an amount of from 0 (zero) to up to 10% (e.g. from 1 to 10%) by weight of the composition (or tablet). The first and second pharmaceutically active components may be the same or different, e.g. the first and second pharmaceutically active components may each comprise (or consist of) irbesartan.

It is to be noted the total amount (i.e. by weight) of (first and second); disintegrant elements in relation to the total composition (or tablet) weight may vary between broad limits, for example from 0.2% to 30% by weight, e.g. the first disintegrant element may be present in an amount of 20% by weight and the second disintegrant element may be present in an amount of 10% by weight.

It is also to be understood herein, that if a “class”, “range”, “group of substances”, etc. is mentioned with respect to a particular characteristic (e.g., temperature, time, particle size, % by weight and the like) of the present invention, the present invention relates to and explicitly incorporates herein each and every specific member and combination of sub-classes, sub-ranges or sub-groups therein whatsoever. Thus, any specified class, range or group is to be understood as a shorthand way of referring to, each and every member of a class, range or group individually as well as each and every possible sub-class, sub-range or sub-group encompassed therein; and similarly with respect to any sub-class, sub-ranges or sub-groups therein. Thus, for example, a pharmaceutical composition is provided herein which may comprise from up to 75% (e.g. from 40 to 75%) by weight of the intra-granular fraction and from up to 60% (e.g. from 25 to 60%) by weight of the extra-granular fraction; it is to be understood herein therefore that the weight percentage of up to 75% includes a specific reference to 60%, 71%, 15 to 55%, 40 to 75%, etc. by weight of the intra-granular fraction; that the weight percentage of up to 60% includes a specific reference to 60%, 51%, 15 to 55%, 40 to 55%, etc. by weight of the extra-granular fraction. As a further example a pharmaceutical composition is provided herein wherein the intra-granular fraction comprises from 0.5 to 20% by weight of the first binder element; it is to be understood herein therefore that the weight percentage of from 0.5 to 20% includes a specific reference to 1%, 11%, 5 to 10%, 4 to 15%, etc. by weight of first binder element.

It is in particular to be understood herein that for any group or range, no matter how defined, a reference thereto is a shorthand way of mentioning and including herein each and every individual member described thereby as well as each and every possible class or sub-group or sub-class of members whether such class or sub-class is defined as positively including particular members, as excluding particular members or a combination thereof; for example an exclusionary definition for a formula, weight percentage, etc., may read as follows: “provided that when one of A and B is −X and the other is Y, −X may not be Z”.

An example pharmaceutical composition (e.g. tablet) in accordance with the present invention is set forth in Table 1 below, where item nos. 1 to 4 are part of the intra-granular fraction and item nos. 5 to 8 are part of the extra-granular fraction:

TABLE 1

| Item No. | | % w/w |
|---|---|---|
| | Intra-granular fraction | |
| | elements | |
| 1 | Irbesartan | 50.00 |
| 2 | Copovidone | 6.67 |
| 3 | Croscarmellose Sodium | 3.33 |
| 4 | Colloidal Silicon Dioxide | 2.00 |
| | Extra-granular fraction | |
| | elements | |
| 5 | Tablettose | 31.97 |
| | (Agglomerated lactose) | |
| 6 | Croscarmellose Sodium | 3.33 |
| 7 | Colloidal Silicon Dioxide | 2.00 |
| 8 | Magnesium Stearate | 0.70 |
| | | 100.0 |

* Milligrams per Irbesartan tablet

For the preparation of the composition as set forth in Table 1 purified water may be used (i.e. as a granulation binder) during the formation of the granules of the intra-granular fraction, i.e. in sufficient amount to promote granulation. In other words the intra-granular fraction may be obtained from a granulation process using an aqueous solvent as a granulation medium.

The present invention also relates to a process for the preparation of a tablet for oral administration, which process may for example comprise the following steps: (i) obtaining an intra-granular fraction by subjecting pharmaceutically acceptable active component and a first pharmaceutically acceptable excipient component to a granulation stage, said, first pharmaceutically acceptable excipient component comprising (or consisting of) a binder element, a first disintegrant element and a first antiadherent element, said intra-granular fraction being at least substantially free of a diluent element and being at least substantially free of a surfactant element; (ii) blending (i.e. dry mixing) granules obtained from step (i) with the elements of an extra-granular fraction comprising a second pharmaceutically acceptable excipient component (and where necessary or desired a further (i.e. second) pharmaceutically acceptable active component), said second pharmaceutically acceptable excipient component comprising (or consisting of) a diluent element (wherein the diluent element may consist of powder, directly compressible grade either spray dried or agglomerated, or the diluent may, optionally or as necessary, be initially (i.e. previously) granulated along with a second binder element), a second disintegrant element, a second anti-adherent element and a lubricant element; and (iii) compressing the blend obtained from step (ii) into tablets. In accordance with the granulating step the granules may be prepared by any suitable (known) manner (e.g. by wet granulation with an aqueous solvent (e.g. water) i.e. without using organic solvents or by any suitable (known) dry granulation technique). Similarly the compression step to form the tablets may also be carried out in any suitable (known) manner.

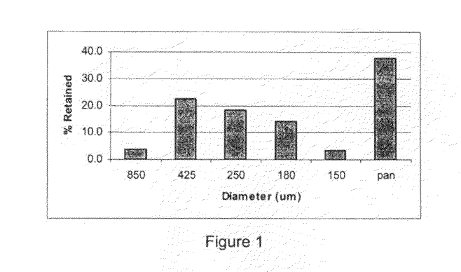

FIG. 1 depicts particle size distribution of a granulate batch of a composition as shown in Table 1 with the data as shown in Table 2 (below).

The granules of the intra-granular fraction may, for example, have a size of from 50 microns to 1.5 mm. A typical particle size distribution of a granule batch is given herein below in Table 2 and depicted in FIG. 1. The weight of the granular material retained on each screen (and in the pan) was determined by obtaining the weight of each screen (and the pan) before screening of the material and weighing, after screening, each screen (and the pan) with the material retained thereon, the difference in weight (i.e. after weight minus before weight) being the weight of the screen oversize material (or in the case of the pan the undersize material or fines).

TABLE 2

| | | Wt of | Wt of | | |
|---|---|---|---|---|---|
| Sieve* | Dia | screen | screen | Sample | % |
| No. | (um) | Before(g) | After(g) | Wt(g) | Retained |
| 20 | 850 | 385.4 | 387.7 | 2.3 | 3.8 |
| 40 | 425 | 355.0 | 368.5 | 13.5 | 22.5 |
| 60 | 250 | 324.0 | 334.9 | 10.9 | 18.2 |
| 80 | 180 | 320.2 | 328.7 | 8.5 | 14.2 |
| 100 | 150 | 319.5 | 321.5 | 2.0 | 3.3 |
| pan | pan | 364.9 | 387.6 | 22.7 | 37.8 |
| | | | Total | 59.9 | 99.8 |

*using (ASTM) E11-70

The “diluent element” (i.e. filler) employed for the extra-granular fraction of the present invention may comprise one or more (known) diluent or filler type substances or compounds which (in appropriate amounts) may be exploited to alter or modify the mass and flow and compression properties of the granules/powder blend required for the desired compression characteristics.

Suitable diluent materials may be selected from the group comprising inorganic phosphates such as dibasic calcium phosphate; sugars such as lactose hydrous or lactose anhydrous mannitol, sorbitol, isomalt; cellulose or cellulose derivatives such as microcrystalline cellulose, silicified microcrystalline cellulose; starch such as pregelatinized starch, as well as mixtures thereof.

The “binder element” (i.e. first or intra-granular binder element) employed for the intra-granular fraction of the present invention may comprise one or more (known) binder type substances or compounds which (in appropriate amounts) may promote the adhesion of (one or more of) the other elements of the intra-granular fraction for the formation of granules by wet granulation. For example, during the granulation process, a non-organic fluid/solvent such as water may be added (in known manner) to a (dry) blend of elements of the intra-granular fraction so as to form larger and/or more free-flowing particles, i.e. granules. Alternatively, the binder element for the intra-granular fraction may be dissolved in water prior to the addition to the dry blend of the other elements of the intra-granular fraction (i.e. for granulation).

Optionally as mentioned herein a “binder element” (i.e. second binder element) may be employed for the extra-granular fraction per se or for the initial granulation of the diluent for the extra-granular fraction. The second binder element of the present invention, if present, may be the same or different from the binder element for the intra-granular element. The second binder element may, for example, comprise one or more (known) binder type substances or compounds which (in appropriate amounts) may promote the adhesion of diluent particles for the formation of diluent granules by wet granulation as noted above.

Suitable binder materials may, for example, be selected from the group comprising starch, povidone, copovidone, hyprollose, hypromellose, polyethylene oxide, sucrose, xanthan gum, polyvinyl alcohol, alginic acid, sodium alginate, guar gum, pullulan, pea starch, and cellulose or cellulose derivatives (such as carboxymethylcellulose sodium, ethylcellulose, hydroxyethyl cellulose, hydroxypropyl cellulose, hydroxypropyl methylcellulose, or methylcellulose, as well as mixtures thereof.

The “disintegrant element” employed for the intra-granular fraction and for the extra-granular fraction of the present invention (i.e. the first and second disintegrant elements) may be the same or different and may comprise one or more (known) disintegrant type substances or compounds which (in appropriate amounts) are capable of facilitating the break up of a tablet prepared from the composition (or formulation) when placed in contact with an aqueous medium (e.g. gastric fluid).

Suitable disintegrant type materials may, for example, be selected from the group comprising crospovidone, starch, starch derivatives (e.g. sodium starch glycollate), croscarmellose sodium, croscarmellose calcium, guar gum, low substituted hydroxypropyl cellulose where the hydroxypropoxyl content may vary in the range from 7% to 9% or from 10% to 12.9% (on a weight by weight basis), as well as mixtures thereof. The quantity of each of the first and second disintegrant elements may, for example as mentioned above vary from 0.1 to 15% by weight of the (tablet) formulation or composition hereby prepared.

The “anti-adherent element” employed for the intra-granular fraction and for the extra-granular fraction of the present invention (i.e. the first and second anti-adherent elements) may be the same or different and may comprise one or more (known) substances or compounds which (in appropriate amounts) are capable of reducing the stickiness of the composition or formulation, for example, inhibiting adherence to metal surfaces.

Suitable anti-adherent type materials may, for example, be selected from the group comprising talc and silicon-containing compounds such as colloidal silicon dioxide as well as mixtures thereof.

The “lubricant element” employed for the extra-granular fraction of the present invention may comprise one or more (known) substances or compounds which (in appropriate amounts) are capable of preventing or inhibiting tablet formation problems, such as those relating to the release of a tablet from the tooling, in compression equipment on which the table is formed; for example, for preventing adherence to the face of the upper punch (picking) or lower punch (sticking) of a tableting apparatus.

Suitable lubricant type materials may, for example, be selected from the group comprising magnesium stearate, calcium stearate, zinc stearate, glyceryl behenate, sodium stearyl fumarate, hydrogenated vegetable oil, hydrogenated castor oil, glyceryl palmitostearate, stearic acid, sucrose fatty acid esters and sodium benzoate as well as mixtures thereof.

As mentioned above, a “surfactant element” for the purposes of the present invention may be any one or more substances or compounds which (in appropriate amounts) are capable of improving or enhancing the wetting and/or solubility characteristics of a drug containing dosage form (e.g. tablet). For example, such surfactant type substances, to be avoided in accordance with the present invention, include substances such as polysorbates, poloxamer, phospholipids, polyoxyethylene castor oil derivatives, sodium lauryl sulphate, dioctyl sodium sulphosuccinate, sucrose fatty acid esters, sorbitan fatty acid esters, polyoxyethylene alkyl ethers, polyethylene glycol-fatty acid esters, tocopheryl polyethylene glycol ester salts, or mixtures thereof.

An example process for the preparation of an example 300 mg irbesartan tablet having the formulation set forth in Table 3 is given below after the Table 3.

TABLE 3

| Item No. | | Function | *mg/tab |
|---|---|---|---|
| | Intra-granular fraction | | |
| | elements | | |
| 1 | Irbesartan | Active/Drug | 300.00 |
| 2 | Plasdone S630 | Binder | 40.00 |
| 3 | Croscarmellose Sodium | Disintegrant | 20.00 |
| 4 | Colloidal Silicon | Anti-adherent | 12.00 |
| | Dioxide | | |
| | Extra-granular | | |
| | fraction elements | | |
| 6 | Agglomerated lactose | Diluent | 191.80 |
| | (directly compressible) | | |
| 7 | Croscarmellose Sodium | Disintegrant | 20.00 |
| 8 | Colloidal Silicon | Anti-adherent | 12.00 |
| | Dioxide | | |
| 9 | Magnesium Stearate | Lubricant | 4.20 |
| | Total Tablet Weight | | 600.0 mg |

*Milligrams per Irbesartan tablet

For the preparation of the composition as set forth in Table 3 purified water may be used (i.e. as a granulation binder) during the formation of the granules of the intra-granular fraction, i.e. in sufficient amount to promote granulation. Again, in other words the intra-granular fraction may be obtained from a granulation process using an aqueous solvent as a granulation medium.

An example preparation process for the manufacture of a 300 mg Irbesartan tablet (referring to Table 3 for the Item number description) is described hereinafter; the process exploits various known types of equipment which are referred to (unless otherwise indicated) by their generic designations.

The example process comprises the following (14) steps or stages:

1) Items 1 and 4 (Table 3) are mixed and sieved together using a comil. The comil consists of a rotating blade that forces the material through a screen that has perforations of specified size (i.e. 0.018 inch perforations).
2) Items 2 and 3 (Table 3) are separately sieved using a vibratory sifter through a screen/mesh of specified opening 20 mesh screen (ASTM) (850 microns).
3) The sieved items 1 and 4 (i.e. undersize material) are loaded, into a high shear granulator. (The granulator comprises a bowl fitted with impeller blades that mix the powders. This enables dry mixing of powders as well as granulating powders when a fluid such as water is admixed with the powders causing lumping or binding.)
4) The sieved items 2 and 3 (i.e. undersize material) are dry mixed with items 1 and 4 in the high shear granulator.
5) Purified water is then added at a fixed rate (600 gm per minute) while mixing with the impellers at fixed speed (200 rpm). After adequate addition of water (35% by weight) and mixing a mass of suitable consistency is obtained.
6) The wet mass is then dried using a fluid bed dryer wherein the wet mass is fluidized using heated air for a predetermined time period.
7) The partially dried material is then milled using the comil and a fixed screen (i.e. 0.187 inch perforations) to break the big lumps into smaller ones to facilitate faster and proper drying.
8) The partially dried milled material is then further dried in the fluid bed dryer.
9) The (completely) dried material is then screened to using a mesh of 850 microns. Any material retained over the mesh is sized to granule size by further milling using a comil.
10) The dried sized granules are then loaded into a blender (i.e. a V or bin type blender).
11) Item 6 (Table 3) is sieved through a mesh size of 850 micron opening. Item 7 (Table 3) and item 8 (Table 3) are mixed and then sieved through a mesh size of 425 micron opening. Item 9 (Table 3) is sieved through a mesh of 425 micron opening.
12) The sieved items 6 to 8 (i.e. undersize material) are then loaded into the blender containing the granules (step 10 above) and the blender is rotated at a fixed speed (14 rpm) for a fixed time (5 minutes)*. *the fixed speed and fixed time may be varied as necessary or desired respectively for example from 10 to 20 rpm and from 1 to fifteen minutes;
13) The sieved item 9 is then loaded into the blender after step 12) and the blender is again rotated at a fixed speed (14 rpm) for a fixed time (2 minute)** and the obtained blend comprising an intra-granular fraction and an extra-granular fraction (i.e. lubricated granules) is unloaded. the fixed speed and fixed time may be varied as necessary or desired respectively for example from 10 to 20 rpm, and from 1 to five minutes.
14) The blend obtained from step 13) is then compressed into tablets using a tablet compression machine fitted with punches and dies of required dimensions.

It is to be understood herein that the above process is provided by way of example only;

In Table 4 which follows the “dissolution performance” as well as the “disintegration performance” of a tablet having a pharmaceutical formulation in accordance with the present invention (namely, formulation no. 1 of Table 4) is compared with that of tablets having other types of pharmaceutical formulations (formulations 2 to 8 of Table 4); all of the formulations were, however, made by compression (see step 14) above) of a formulation comprising an intra-granular fraction intermingled with an extra-granular fraction as set forth in Table 4.

The test for “disintegration performance” was performed to determine the time taken for the dosage form (i.e. tablet) to disintegrate completely in water maintained at 37 degrees centigrade (approximately body temperature). The apparatus used is described in the United States Pharmacopoeia (USP 29/NF24 published by U.S. Pharmacopoeia Convention Inc. 2006) under disintegration test monograph (701) and consisted of cylindrical tubes with a bottom mesh; the apparatus was as Erweka model ZP 502, Erweka GmbH, Germany. The assembly of 6 tubes each of which holds a tablet is immersed in a beaker containing water. Then entire assembly bobs up and down in the water medium at a fixed rate and the time (32 cycles per minute) taken for complete disintegration is noted (i.e. when no visible mass is retained over the bottom mesh of the cylindrical tubes).

The test results for “dissolution performance” of a tablet, as used herein with respect to irbesartan, refers to the weight % of dissolved irbesartan, based on the total weight of irbesartan contained in the tablet after a specified time under specific conditions. For example, for an irbesartan tablet containing 300 mg of irbesartan, a dissolution value of 95% indicates that about 285 mg of irbesartan has dissolved.

For the dissolution tests, the dissolution apparatus using USP apparatus 2 as described in the United States Pharmacopoeia (USP 29/NF24 published by U.S. Pharmacopoeia Convention Inc. 2006) under dissolution testing monograph (711) was used, where a tablet is introduced into a vessel containing 0.1 N hydrochloric acid maintained at 37 degrees centigrade (approximately body temperature) and the contents stirred with a paddle rotating at 50 rpm. The dissolution apparatus used was a Varian VK 7000, Vankel Technologies Group, USA. Periodically, samples are drawn and filtered. The filtered solution sample was then analyzed for dissolved irbesartan in known manner.

TABLE 4

| | | Percentage of Tablet | | | | | | | |
|---|---|---|---|---|---|---|---|---|---|
| | | Formu- | | | | | | | |
| | | lation | | | | | | | |
| | | no. 1 (i.e. | Formu- | Formu- | Formu- | Formu- | Formu- | Formu- | Formu- |
| | | of present | lation | lation | lation | lation | lation | lation | lation |
| Ingredient | Function | invention) | no. 2 | no. 3 | no. 4 | no. 5 | no. 6 | no. 7 | no. 8 |
| Intra-granular | | | | | | | | | |
| fraction | | | | | | | | | |
| elements | | | | | | | | | |
| Irbesartan | Active | 50.0 | 50.0 | 50.0 | 50.0 | 50.0 | 50.0 | 50.0 | 50.0 |
| Pregelatinized | Binder | — | 5.0 | 5.0 | 5.0 | 5.0 | 5.0 | — | — |
| Starch | | | | | | | | | |
| Croscarmellose | Disintegrant | 3.3 | 1.0 | — | 1.0 | 1.0 | 1.0 | 1.0 | 3.3 |
| Sodium | | | | | | | | | |
| Crospovidone | Disintegrant | — | — | 3.0 | — | — | — | — | — |
| XL | | | | | | | | | |
| Colloidal Silicon | Anti- | 2.0 | 2.0 | 2.0 | 2.0 | 2.0 | 2.0 | 2.0 | 2.0 |
| Dioxide | adherent | | | | | | | | |
| Microcrystalline | Diluent | — | 16.0 | 14.5 | 15.0 | 14.5 | 14.5 | — | — |
| Cellulose PH01 | | | | | | | | | |
| Povidone k30 | Binder | — | 4.0 | 4.0 | 4.0 | 4.0 | 4.0 | 2.0 | — |
| Plasdone S630 | Binder | 6.7 | — | — | — | — | — | — | 6.7 |
| Sucrose Fatty | Surfactant | — | — | — | — | 5.0 | — | 2.0 | — |
| acid ester F160 | | | | | | | | | |
| Docusate | Surfactant | — | — | 2.0 | — | — | — | — | — |
| Sodium | | | | | | | | | |
| Sodium lauryl | Surfactant | — | — | — | — | — | — | — | 0.5 |
| sulfate | | | | | | | | | |
| PEG 400 | Solubilizer | — | — | — | 3.0 | — | 5.0 | — | — |
| Extra-granular | | | | | | | | | |
| fraction | | | | | | | | | |
| elements | | | | | | | | | |
| Microcrystalline | Diluent | — | 13.0 | 16.2 | 15.0 | 13.5 | 13.5 | — | 31.2 |
| Cellulose | | | | | | | | | |
| PH102 | | | | | | | | | |
| Pregelatinized | Binder/ | — | — | — | — | — | — | 5.0 | — |
| starch | Disintegrant | | | | | | | | |
| Sodium lauryl | Surfactant | — | 1.0 | — | — | — | — | — | — |
| sulfate | | | | | | | | | |
| Croscarmellose | Disintegrant | 3.3 | 2.0 | — | 2.0 | 2.0 | 2.0 | 3.0 | 3.3 |
| Sodium | | | | | | | | | |
| Colloidal Silicon | Anti- | 2.0 | 2.0 | 2.0 | 2.0 | 2.0 | 2.0 | 2.0 | 2.0 |
| Dioxide | Adherent | | | | | | | | |
| Crospovidone | Disintegrant | — | — | 3.0 | — | — | — | — | — |
| XL | | | | | | | | | |
| Magnesium | Lubricant | 1.0 | 1.0 | 1.0 | 1.0 | 1.0 | 1.0 | 1.0 | 1.0 |
| Stearate | | | | | | | | | |
| Tablettose | Diluent | 31.7 | — | — | — | — | — | 32.0 | — |
| (agglomerated | | | | | | | | | |
| lactose) | | | | | | | | | |
| Disintegration | | 3 minutes | 7 minutes | 13 minutes | 2 minutes | >10 min. | 2-3 minutes | 2 minutes | 3 minutes |
| Time | | 15 seconds | 20 seconds | | 15 seconds | Approx. | | | 10 seconds |
| | | | | | | 10-11 minutes | | | |
| | Time | | | | | | | | |
| Dissolution | (minutes) | Average % of Drug Released | | | | | | | |
| | 5 | 99 | 48 | — | 47 | 20 | 47 | 52 | 52 |
| | 10 | 100 | 63 | — | 61 | 37 | 63 | 63 | 58 |
| | 15 | 100 | 73 | — | 68 | 52 | 72 | 73 | 67 |
| | 20 | 101 | 77 | — | 71 | 63 | 77 | 81 | 74 |
| | 30 | 101 | 83 | — | 78 | 77 | 85 | 88 | 79 |
| | 45 | — | — | — | — | — | — | 95 | 91 |

As may be seen from Table 4, the tablet having formulation 1 (in accordance with the present invention) has a dissolution characteristic such that total dissolution occurs after only about 10 minutes; the other tablets (of formulations 2 to 8) on the other hand exhibit dissolution of less than 70% by weight after about 10 minutes.

1. A pharmaceutical composition comprising an intra-granular fraction intermingled with an extra-granular fraction, said intra-granular fraction consisting of granules comprising a pharmaceutically acceptable active component and a first pharmaceutically acceptable excipient component said first pharmaceutically acceptable excipient component comprising a first binder element, a first disintegrant element and a first antiadherent element, said intra-granular fraction being at least substantially free of a diluent element and being at least substantially free of a surfactant element, said extra-granular fraction comprising a second pharmaceutically acceptable excipient component, said second pharmaceutically acceptable excipient component comprising a diluent element, a second disintegrant element, a second anti-adherent element and a lubricant element said extra-granular fraction being at least substantially free of a surfactant element.

2. A pharmaceutical composition as defined in claim 1 wherein the pharmaceutically acceptable active component of said intra-granular fraction comprises a member selected from the group consisting of irbesartan and pharmaceutically acceptable salts thereof.

3. A pharmaceutical composition as defined in claim 2 wherein said first pharmaceutically acceptable excipient component consists of the first binder element, the first disintegrant element and the first antiadherent element.

4. A pharmaceutical composition as defined in claim 3 wherein said second pharmaceutically acceptable excipient component consists of the diluent element, the second disintegrant element, the second antiadherent element and the lubricant.

5. A pharmaceutical composition as defined in claim 4 wherein said first binder element is selected from the group comprising starch, povidone, copovidone, hyprollose, hypromellose, polyethylene oxide, sucrose, xanthan gum, polyvinyl alcohol, alginic acid, sodium alginate, guar gum, pullulan, pea starch, and cellulose, carboxymethylcellulose sodium, ethylcellulose, hydroxyethyl cellulose, hydroxypropyl cellulose, hydroxypropyl methylcellulose, methylcellulose and mixtures thereof.

6. A pharmaceutical composition as defined in claim 5 wherein said first disintegrant element is selected from the group comprising crospovidone, starch, sodium starch glycollate, croscarmellose sodium, croscarmellose calcium, guar gum, and mixtures thereof and said first antiadherent element is selected from the group comprising talc, colloidal silicon dioxide and mixtures thereof.

7. A pharmaceutical composition as defined in claim 3 wherein said second pharmaceutically acceptable excipient component comprises a second binder element.

8. A pharmaceutical composition as defined in claim 7 wherein said second pharmaceutically acceptable excipient component consists of the diluent element, the second binder element, the second disintegrant element, the second antiadherent element and the lubricant.

9. A pharmaceutical composition as defined in claim 8 wherein said first binder element and said second binder element are independently selected from the group comprising starch, povidone, copovidone, hyprollose, hypromellose, polyethylene oxide, sucrose, xanthan gum, polyvinyl alcohol, alginic acid, sodium alginate, guar gum, pullulan, pea starch, cellulose, carboxymethylcellulose sodium, ethylcellulose, hydroxyethyl cellulose, hydroxypropyl cellulose, hydroxypropyl methylcellulose, methylcellulose and mixtures thereof.

10. A pharmaceutical composition as defined in claim 9 wherein said first disintegrant element is selected from the group comprising crospovidone, starch, sodium starch glycollate, croscarmellose sodium, croscarmellose calcium, guar gum, and mixtures thereof and said first antiadherent element is selected from the group comprising talc, colloidal silicon dioxide and mixtures thereof.

11. A pharmaceutical composition as defined in claim 6 wherein said diluent element is selected from the group comprising dibasic calcium phosphate; lactose hydrous, lactose anhydrous, mannitol, sorbitol, isomalt, cellulose, microcrystalline cellulose, silicified microcrystalline cellulose, pregelatinized starch and mixtures thereof, said second disintegrant element is selected from the group comprising crospovidone, starch, sodium starch glycollate, croscarmellose sodium, croscarmellose calcium, guar gum, and mixtures thereof said second antiadherent element is selected from the group comprising talc, colloidal silicon dioxide and mixtures thereof, and said lubricant element is selected from the group comprising magnesium stearate, calcium stearate, zinc stearate, glyceryl behenate, sodium stearyl fumarate, hydrogenated vegetable oil, hydrogenated castor oil, glyceryl palmitostearate, stearic acid, sodium benzoate and mixtures thereof.

12. A pharmaceutical composition as defined in claim 11 wherein said diluent element is selected from the group comprising dibasic calcium phosphate; lactose hydrous, lactose anhydrous, mannitol, sorbitol, isomalt, cellulose, microcrystalline cellulose, silicified microcrystalline cellulose, pregelatinized starch and mixtures thereof, said second disintegrant element is selected from the group comprising crospovidone, starch, sodium starch glycollate, croscarmellose sodium, croscarmellose calcium, guar gum, and mixtures thereof said second antiadherent element is selected from the group comprising talc, colloidal silicon dioxide and mixtures thereof, and said lubricant element is selected from the group comprising magnesium stearate, calcium stearate, zinc stearate, glyceryl behenate, sodium stearyl fumarate, hydrogenated vegetable oil, hydrogenated castor oil, glyceryl palmitostearate, stearic acid, sodium benzoate and mixtures thereof.

13. A pharmaceutical composition as, defined in claim 11 wherein said pharmaceutical composition comprises from 40 to 75% by weight of said intra-granular fraction and from 25 to 60% by weight of said extra-granular fraction.

14. A pharmaceutical composition as defined in claim 12 wherein said pharmaceutical composition comprises from 40 to 75% by weight of said intra-granular fraction and from 25 to 60% by weight of said extra-granular fraction.

15. A pharmaceutical composition as defined in claim 13 wherein said intra-granular fraction comprises a first pharmaceutically acceptable active component and said extra-granular fraction comprises a second pharmaceutically acceptable active component, said composition comprising from 30 to 71% by weight of said first pharmaceutically acceptable active component and from 0 to 10% by weight of said second pharmaceutically acceptable active component and wherein said first and second pharmaceutically acceptable excipient components together comprise from 29 to 70% by weight of said composition.

16. A pharmaceutical composition as defined in claim 15 wherein said composition comprises from 0.5 to 20% by weight of said first binder element, from 0.1 to 15% by weight of said first disintegrant element, from 0.1 to 5% by weight of said first anti-adherent element, from 10 to 59.6% by weight of said diluent, from 0.1 to 15% by weight of said second disintegrant element, from 0.1 to 5% by weight of said second anti-adherent element and from 0.2 to 7.5% by weight of said lubricant element.

17. A pharmaceutical composition as defined in claim 14 wherein said intra-granular fraction comprises a first pharmaceutically acceptable active component and said extra-granular fraction comprises a second pharmaceutically acceptable active component, said composition comprising from 30 to 71% by weight of said first pharmaceutically acceptable active component and from 0 to 10% by weight of said second pharmaceutically acceptable active component and wherein said first and second pharmaceutically acceptable excipient components together comprise from 29 to 70% by weight of said composition.

18. A pharmaceutical composition as defined in claim 17 wherein said composition comprises from 0.5 to 20% by weight of said first binder element, from 0.1 to 15% by weight of said first disintegrant element, from 0.1 to 5% by weight of said first anti-adherent element, from 10 to 59.6% by weight of said diluent, from 0.5 to 10% by weight of said second binder element, from 0.1 to 15% by weight of said second disintegrant element, from 0.1 to 5% by weight of said second anti-adherent element and from 0.2 to 7.5% by weight of said lubricant element.

19. A pharmaceutical composition as defined in claim 18 wherein both the first and second pharmaceutically acceptable active components comprise a member selected from the group consisting of irbesartan and pharmaceutically acceptable salts thereof.

20. A pharmaceutical, composition as defined in claim 14 wherein the pharmaceutically acceptable active component of said intra-granular fraction consists of irbesartan.

21. A pharmaceutical composition as defined in claim 18 wherein said first and second pharmaceutically acceptable active components each consists of irbesartan.

22. A pharmaceutical composition as defined in claim 21 wherein said intra-granular fraction has been obtained from a granulation process using an aqueous solvent as a granulation medium.
